SEQUENCE LISTING (64 sequences)

SEQ ID NO: 1 - DNA, 20 bp, Homo sapiens
auucacuuucauaaugcugg

SEQ ID NO: 2 - DNA, 25 bp, Homo sapiens
guaagauucacuuucauaaugcugg

SEQ ID NO: 3 - DNA, 23 bp, Homo sapiens
gauagcuauauauagauagcuuu

SEQ ID NO: 4 - DNA, 26 bp, Homo sapiens
auagauagcuauauauagauagcuuu

SEQ ID NO: 5 - DNA, 20 bp, Homo sapiens
gauagcuauauauagauagc

SEQ ID NO: 6 - DNA, 20 bp, Homo sapiens
auagauauagauagcuauau

SEQ ID NO: 7 - DNA, 23 bp, Homo sapiens
auuaaccuuuuaucuaauaguuu

SEQ ID NO: 8 - DNA, 26 bp, Homo sapiens
auuaaccuuuuaucuaauaguuuugg

SEQ ID NO: 9 - DNA, 20 bp, Homo sapiens
aauaguuuuggcaucaaaau

SEQ ID NO: 10 - DNA, 23 bp, Homo sapiens
aauaguuuuggcaucaaaauucu

SEQ ID NO: 11 - DNA, 26 bp, Homo sapiens
aauaguuuuggcaucaaaauucuuua

SEQ ID NO: 12 - DNA, 20 bp, Homo sapiens
ucuaauaguuuuggcaucaa

SEQ ID NO: 13 - DNA, 23 bp, Homo sapiens
ucuaauaguuuuggcaucaaaau

SEQ ID NO: 14 - DNA, 20 bp, Homo sapiens
gaucugucugaucguuucuu

SEQ ID NO: 15 - DNA, 25 bp, Homo sapiens
aucuucuauaacgcuucacauucca

SEQ ID NO: 16 - DNA, 25 bp, Homo sapiens
ucuauaacgcuucacauuccagauc

SEQ ID NO: 17 - DNA, 25 bp, Homo sapiens
cuucuauaacgcuucacauuccaga

SEQ ID NO: 18 - DNA, 20 bp, Homo sapiens
uuuguuucacaagacauuuu

SEQ ID NO: 19 - DNA, 25 bp, Homo sapiens
accuuccuucuuuuugauuuugucu

SEQ ID NO: 20 - DNA, 30 bp, Homo sapiens
cuggcagacuuacuccuuaauuuaaggaau

SEQ ID NO: 21 - DNA, 21 bp, Homo sapiens
uccuucuuuuugauuuugucu

SEQ ID NO: 22 - DNA, 20 bp, Homo sapiens
caccuuccuucuuuuugauu

SEQ ID NO: 23 - DNA, 25 bp, Mus musculus
acuuuccuucuuuuuuauuuugucu

SEQ ID NO: 24 - DNA, 30 bp, Mus musculus
augacagacuuacuucuuaauuuguaugug

SEQ ID NO: 25 - DNA, 30 bp, Mus musculus
uuuaaaaugacagacuuacuucuuaauuug

SEQ ID NO: 26 - DNA, 30 bp, Mus musculus
ugagcacuuuccuucuuuuuuauuuugucu

SEQ ID NO: 27-28 - skipped (2 entries)

SEQ ID NO: 29 - DNA, 20 bp, Homo sapiens
acaacuuugggaggcggagg

SEQ ID NO: 30 - DNA, 20 bp, Homo sapiens
aaucccacaacuuugggagg

SEQ ID NO: 31 - DNA, 26 bp, Homo sapiens
gcucaugccuacaaucccacuucuuu

SEQ ID NO: 32 - DNA, 23 bp, Homo sapiens
gcucaugccuacaaucccacuuc

SEQ ID NO: 33 - DNA, 20 bp, Homo sapiens
gcucaugccuacaaccccac

SEQ ID NO: 34 - DNA, 20 bp, Homo sapiens
gcaguggcucaugccuacaa

SEQ ID NO: 35 - DNA, 20 bp, Homo sapiens
uuugguuuucuugcaguggc

SEQ ID NO: 36 - DNA, 20 bp, Homo sapiens
caauuauuaggcugcaguua

SEQ ID NO: 37 - DNA, 20 bp, Homo sapiens
tatcccaaagaaaacaatta

SEQ ID NO: 38 - DNA, 20 bp, Homo sapiens
uuuuaauguacuuuaaaagu

SEQ ID NO: 39 - DNA, 20 bp, Homo sapiens
auagucuuuuaauguacuuu

SEQ ID NO: 40 - DNA, 20 bp, Homo sapiens
uaugaucagaaauuaaguug

SEQ ID NO: 41 - DNA, 20 bp, Homo sapiens
uauucaacaaaauaugauca

SEQ ID NO: 42 - DNA, 25 bp, Homo sapiens
uuuuggcaucaaaauucuuuaauau

SEQ ID NO: 43 - DNA, 25 bp, Homo sapiens
aauaguuuuggcaucaaaauucuuu

SEQ ID NO: 44 - DNA, 28 bp, Homo sapiens
ucuaauaguuuuggcaucaaaauucuuu

SEQ ID NO: 45 - DNA, 30 bp, Homo sapiens
ccuuuuaucuaauaguuuuggcaucaaaau

SEQ ID NO: 46 - DNA, 30 bp, Homo sapiens
auuaaccuuuuaucuaauaguuuuggcauc

SEQ ID NO: 47 - DNA, 25 bp, Homo sapiens
auuaaccuuuuaucuaauaguuuug

SEQ ID NO: 48 - DNA, 20 bp, Homo sapiens
auuaaccuuuuaucuaauag

SEQ ID NO: 49 - DNA, 23 bp, Homo sapiens
uagauuaaccuuuuaucuaauag

SEQ ID NO: 50 - DNA, 26 bp, Homo sapiens
auguagauuaaccuuuuaucuaauag

SEQ ID NO: 51 - DNA, 20 bp, Homo sapiens
gaauucuaguagggauguag

SEQ ID NO: 52 - DNA, 20 bp, Homo sapiens
aaaauggcaucauauccuaa

SEQ ID NO: 53 - DNA, 25 bp, Homo sapiens
gauauaaaauggcaucauauccuaa

SEQ ID NO: 54 - DNA, 20 bp, Homo sapiens
uauaaaauggcaucauaucc

SEQ ID NO: 55 - DNA, 20 bp, Homo sapiens
gauauaaaauggcaucauau

SEQ ID NO: 56 - DNA, 29 bp, Homo sapiens
ggauguagauuaacuuacauuaaccuuuc

SEQ ID NO: 57 - DNA, 23 bp, Homo sapiens
aauaguuuuggcuucaaaauucu

SEQ ID NO: 58 - DNA, 23 bp, Homo sapiens
aauaguuuaggcaucaauauucu

SEQ ID NO: 59 - DNA, 23 bp, Homo sapiens
aauaguuuuggcaucaaaauuuu

SEQ ID NO: 60 - DNA, 23 bp, Homo sapiens
aauaguuuuggcuucaaaauuuu

SEQ ID NO: 61 - DNA, 20 bp, Homo sapiens
uggagcuugacaccacccug

SEQ ID NO: 62 - DNA, 20 bp, Homo sapiens
acuugagaccuggagcuuga

SEQ ID NO: 63 - DNA, 20 bp, Homo sapiens
uagggggaucacuugagacc

SEQ ID NO: 64 - DNA, 20 bp, Homo sapiens
gaggcggagguagggggauc

(19) United States
(12) Patent Application Publication
(10) Pub. No.: US 2017/0051277 A1
(43) Pub. Date: Feb. 23, 2017

(54) ANTISENSE OLIGOMERS AND METHODS FOR TREATING SMN-RELATED PATHOLOGIES

(71) Applicant: THE UNIVERSITY OF WESTERN AUSTRALIA, Nedland, Western Australia (AU)

(72) Inventors: Stephen Donald Wilton, Applecross (AU); Sue Fletcher, Bayswater (AU); Loren Louise Price, Wembley (AU); Chalermchai Mitrpant, Bangkok (TH)

(21) Appl. No.: 15/021,642

(22) PCT Filed: Sep. 12, 2014
(86) PCT No.: PCT/AU2014/000907

§ 371 Date: Mar. 11, 2016

(30) Foreign Application Priority Data
Sep. 13, 2013 (AU) 2013903524

(51) Int. Cl. C12N 15/113

(52) U.S. Cl. CPC C12N 15/113; C12N 2310/11; C12N 2310/31; C12N 2310/3233

(57) ABSTRACT

An antisense oligonucleotide of 10 to 50 nucleotides comprising a targeting sequence complementary to a region near or within intron 6, intron 7, or exon 8 of the Survival Motor Neuron 2 (SMN2) gene pre-mRNA.

### FIELD OF THE INVENTION

The present invention relates to antisense oligomers and compositions suitable for facilitating splice modification, in particular in Survival Motor Neuron (SMN) gene transcripts. The invention also provides methods for inducing exon inclusion in these gene transcripts using the antisense oligomers of the invention, as well as therapeutic compositions adapted for use in the methods of the invention.

### BACKGROUND ART

The following discussion of the background art is intended to facilitate an understanding of the present invention only. The discussion is not an acknowledgement or admission that any of the material referred to is or was part of the common general knowledge as at the priority date of the application.

Spinal muscular atrophy (SMA), the second most common autosomal recessive disorder in humans, is the leading genetic cause of death in children under the age of 2 years. The overall incidence of SMA is in the order of 1 in 10,000 live births, with a carrier frequency of 1/35 (Prior, 2004). Three forms of SMA are recognised, with type I SMA being the most severe and type III SMA at the milder end of the scale. Children affected with SMA I never sit and usually die within the first year of life, whereas those with SMA III acquire the ability to walk, and have a normal life expectancy. An intermediate form (SMA II), describes those individuals who can sit unsupported but will never walk.

SMA is caused most commonly by genomic deletions of the Survival Motor Neuron 1 (SMN1) gene on chromosome 5, resulting in the loss of functional SMN protein. SMN is ubiquitously expressed and is mainly localized in the cytoplasm and nuclear “gems” (Gemini of coiled bodies). Multiple components have been identified as interacting with SMN, indicating its involvement in various cellular processes including transcription, pre-mRNA splicing, mRNA transport and the assembly of ribonuclear protein particles (RNPs) (Gubitz, 2004; Hua, 2004; Meister, 2001; Pellizzoni, 2002).

The SMN genes span over 25 kb and are processed into a 1.62 kb full-length SMN transcript (SMN-FL), or a shorter transcript missing exon 7 (SMNΔ7). Although humans have extra copies of the SMN gene, the centromeric SMN2 gene (or genes) cannot adequately compensate for SMN1 loss unless present at high copy number, since the majority of SMN2 gene transcripts lack exon 7 due to non-productive splicing.

The human SMN1 and SMN2 genes only differ by a handful of nucleotide substitutions: two are contained within exons, neither of which alters the coding sequence and eight changes occur in the introns (Monani, 1999). While these two genes could potentially encode identical proteins, a C>T polymorphism near the beginning of SMN2 exon 7 is regarded as one of the major factors leading to the loss of the exon from most SMN2 gene transcripts (FIG. 1). It now appears that a combination of the abrogation of an enhancer (SF2/ASF) necessary for exon 7 definition, with the concomitant creation of a silencer element, leads to suppression of exon 7 recognition and its subsequent removal from the mature mRNA with introns 6 and 7.

In addition, other silencer motifs have been identified in exon 7 (Hua, 2007), and introns 6 and 7 (Singh, 2007; Hua, 2008), with predicted weak exon 7 acceptor and donor sites also thought to predispose this exon to omission from the mature transcript (Lim, 2001). The SMNΔ7 mRNA is translated into a truncated non-functional protein, which is unstable and cannot self-associate, a property that correlates with disease severity.

In neuronal cells, 90% of the normally expressed SMN2 transcripts are missing exon 7 and the residual 10% of SMN2 transcripts containing exon 7 are insufficient to provide motor neuronal protection in the absence of SMN1, although the level of protein seems to protect most other tissues, including other neuronal populations. Increased copies of SMN2 lead to higher levels of full-length SMN transcript and protein, which modulates the severity of the phenotype. Indeed, humans missing SMN1, but carrying 5-6 copies of SMN2 have been reported to be normal (Swoboda, 2005), while 8 copies of SMN2 rescues the phenotype in SMN−/− mice (Monani, 2000).

Although several studies have identified antisense oligomers (AOs) that have had some small effect on the levels of SMN, many of the AOs tested had either no effect on exon skipping or promoted counter-productive exon 7 skipping (Hua 2007; Hua 2008; Lim 2001; Madocsai, 2005).

It is against this background that therapies for the treatment of muscular atrophies, including spinal muscular atrophy, are sought to be developed.

### SUMMARY OF THE INVENTION

Broadly, according to one aspect of the invention, there is provided an isolated or purified antisense oligomer for modifying pre-mRNA splicing in an SMN gene transcript or part thereof. Preferably, there is provided an isolated or purified antisense oligomer for inducing exon inclusion in an SMN gene transcript or part thereof. For example, in one aspect of the invention, there is provided an antisense oligonucleotide of 10 to 50 nucleotides comprising a targeting sequence complementary to a region near or within intron 6, intron 7, or exon 8 of the Survival Motor Neuron 2 (SMN2) gene pre-mRNA. Preferably, the antisense oligonucleotide is a phosphorodiamidate morpholino oligomer.

Preferably, the antisense oligomer is selected from the group comprising the sequences set forth in Tables 1, 2 and 5 to 7, bar SEQ ID NO:s 1 to 6.

The SMN gene may be SMN2. The target site may be a silencer site in the SMN2 gene transcript.

Accordingly, in one embodiment, the antisense oligomer may be an antisense oligomer capable of inducing inclusion of exon 7 of the SMN2 gene. As such, the antisense oligomer may be a molecule directed towards a target sequence in intron 6, intron 7, or exon 8 of SMN2 pre-mRNA. Preferably, the antisense oligomer is selected from the group comprising the sequences set forth in Tables 1, 2 and 5 to 7 bar SEQ ID NOs 1 to 6.

The antisense oligomer may be an antisense oligomer capable of inducing exon 7 inclusion and, if desired, intron 7 inclusion in mature SMN2 mRNA. As such, the antisense oligomer of the invention may be used to generate SMN2 transcripts containing exon 7 and intron 7, wherein exon 7 contains the normal termination codon, and intron 7 becomes part of the 3′ UTR of the SMN gene transcript. Preferably, such antisense oligomers are chosen from those in Table 2.

The antisense oligomer of the invention may be selected to be an antisense oligomer capable of binding to a selected SMN target site, wherein the target site is an mRNA splicing site selected from a splice donor site, splice acceptor sites, or exonic splicing elements.

According to a still further aspect of the invention, there is provided one or more antisense oligomers as described herein for use in an antisense oligomer-based therapy.

More specifically, the antisense oligomer may be selected from the group consisting of any one or more of SEQ ID NOs: 7 to 17 and 29 to 64, specifically SEQ ID NOs: 7 to 13, 29, 44, 53 and 54, more specifically SEQ ID NOs: 7 to 13, most specifically SEQ ID NO. 10, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in an SMN gene transcript.

The invention extends also to a combination of two or more antisense oligomers capable of binding to a selected target to induce exon inclusion in an SMN gene transcript, including a construct comprising two or more such antisense oligomers.

Advantageously, the invention also provides a method for enhancing or modulating SMN2 intron 7 and exon 7 inclusion in a transcript, the method including the step of using one or more antisense oligomers of the invention. Similarly, the inventions also provides a method of treating Spinal muscular atrophy (SMA) or a condition associated with SMA in a subject in need thereof, comprising administering to the subject an effective amount of an antisense oligonucleotide of 10 to 50 nucleotides comprising a targeting sequence complementary to a region near or within intron 6, intron 7, or exon 8 of the Survival Motor Neuron 2 (SMN2) gene pre-mRNA. Preferably, the antisense oligonucleotide is a phosphorodiamidate morpholino oligomer.

According to another aspect of the invention, there is provided a method of treating an SMN-related pathology in a patient, comprising the step of:
a) administering to the patient a composition comprising one or more antisense oligomers as described herein.

The invention further provides a pharmaceutical, prophylactic, or therapeutic composition for the treatment of an SMN-related pathology in a patient, the composition comprising:
a) one or more antisense oligomers as described herein, and
b) one or more pharmaceutically acceptable carriers and/or diluents.

The composition may comprise about 1 nM to 1000 nM of each of the desired antisense oligomer(s) of the invention. Preferably, the composition may comprise about 10 nM to 500 nM, most preferably between 1 nM and 10 nM of each of the antisense oligomer(s) of the invention

According to another aspect of the invention there is provided the use of one or more antisense oligomers as described herein in the manufacture of a medicament for the modulation or control of SMN-related pathologies. There is therefore provided the use of an effective amount of an antisense oligonucleotide of 10 to 50 nucleotides comprising a targeting sequence complementary to a region near or within intron 6, intron 7, or exon 8 of the Survival Motor Neuron 2 (SMN2) gene pre-mRNA for the manufacture of a medicament for the treatment of Spinal muscular atrophy (SMA) or a condition associated with SMA. Preferably, the antisense oligonucleotide is a phosphorodiamidate morpholino oligomer.

The SMN-related pathology may be a muscular atrophy, such as Spinal Muscular Atrophy (SMA) arising from loss of a functional SMN gene product.

According to yet another aspect of the invention, there is provided a kit comprising at least one antisense oligomer as described herein, a suitable carrier, and instructions for its use.

The present invention further provides one or more antisense oligomers adapted to aid in the prophylactic or therapeutic treatment of a genetic disorder such as an SMN-related pathology in a form suitable for delivery to a patient.

According to another aspect, the invention provides a method for treating a patient suffering from a genetic disease or pathology wherein there is a deleterious mutation in an SMN gene and the effect of the mutation can be abrogated by splice manipulation, comprising the steps of:
a) selecting one or more antisense oligomers as described herein; and
b) administering the antisense oligomers to the patient.

The invention also provides for the use of purified and isolated antisense oligomers as described herein, for the manufacture of a medicament for treatment of an SMN-related genetic disease.

The invention extends, according to a still further aspect thereof, to cDNA or cloned copies of the antisense oligomer sequences of the invention, as well as to vector containing the antisense oligomer sequences of the invention. The invention extends further also to cells containing such sequences and/or vectors.

The patient may be a mammal, including a human.

The invention further provides a method of treating a condition characterised by incorrect SMN expression in a patient, particularly a condition associated with SMA, comprising the step of:
a) administering to the patient an effective amount of one or more antisense oligomers as described herein.

Preferably, the antisense oligomers administered are relevant to the particular genetic lesion in that patient that led to the incorrect SMN expression in the patient.

Furthermore, the invention provides a method for prophylactically treating a patient to prevent or at least minimise SMA, comprising the step of:
a) administering to the patient an effective amount of one or more antisense oligomers or pharmaceutical composition comprising one or more antisense oligomers.

The invention further provides a method for manipulating splicing in an SMN gene transcript, the method including the step of:
a) providing one or more of the antisense oligomers as described herein and allowing the oligomer(s) to bind to a target nucleic acid site.

According to a still further aspect of the invention, there is provided a method of treating a patient with a pathology caused by incorrect, incomplete, or defective SMN-splicing, the method including the step of:
a) administering to the patient an effective amount of one or more antisense oligomers as described herein or a composition as described herein.

According to yet another aspect of the invention, there is provided a splice manipulation target nucleic acid sequence for SMN comprising the DNA equivalents of the nucleic acid sequences set forth in Tables 1, 2 and 5 to 7 bar SEQ ID NOs 1 to 6. The sequences are preferably selected from the group consisting of SEQ ID NOs: 7 to 17 and 29 to 64, preferably SEQ ID NOs: 7 to 13, 29, 44, 53 and 54, most preferably SEQ ID NO: 10, and sequences complementary thereto.

Further aspects of the invention will now be described with reference to the accompanying non-limiting examples and drawings.

### BRIEF DESCRIPTION OF THE DRAWINGS

In the drawings:

FIG. 1 shows a diagrammatic representation of SMN1 and SMN2 splicing patterns. indicating predominant alternative splicing of the SMN genes in response to the C (SMN1) or T (SMN2) polymorphism in exon 7. SMN1 gene expression typically results in 90% of transcripts containing exon 7 (SMN-FL) and only 10% of transcripts skipping exon 7 SMNΔ7. SMN2 expression, in the absence of an SMN1 gene, has compromised exon 7 recognition. Approximately equal amounts of these transcripts are generated in SMA patient fibroblasts, but only ˜10% SMN-FL: 90% SMNΔ7 mRNA is observed in neuronal cells from SMA patients.

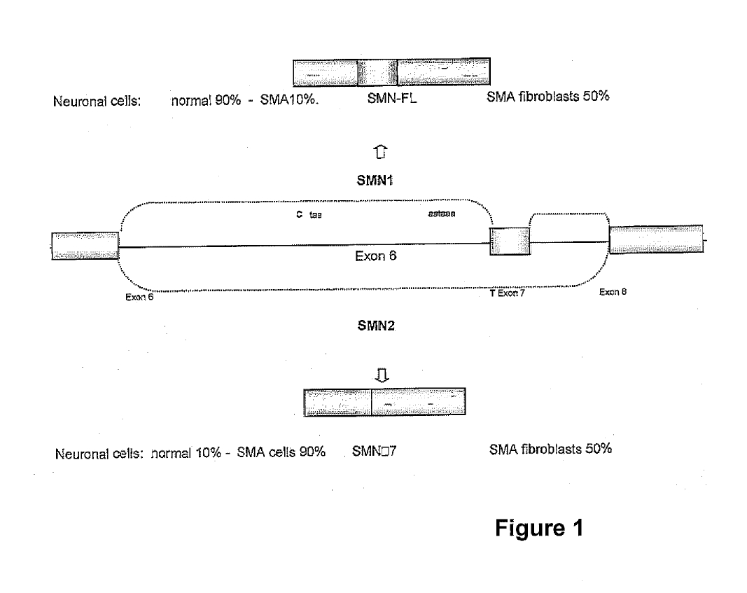

FIG. 2 shows RT-PCR studies indicating changes in the amounts of SMN-FL and SMNΔ7 RNA (indicated with arrow) in SMA patient fibroblasts after transfection with oligomers directed to intron 6 (AO SEQ ID NOs: 3-6) and intron 7 (AO SEQ ID NOs: 7-13) at 50, 25, 10 and 5 nM. A clear dose dependent response is seen with AO SEQ ID NOs: 6, 10 and 13. An oligomer (SEQ ID NO. 1) described by Singh et al. 2006 (Singh, 2006 #74) is indicated (Ctrl AO) and used as a positive control.

FIG. 3 shows RT-PCR results of AOs designed to induce exon skipping in normal human cells, to develop an SMA cell, and eventually a transient mouse model of SMA. AO SEQ ID NOs: 18, 19 and 20 show better levels of skipping when in combination, shown at 100, 50, 25 and 12.5 nM. The single AO SEQ ID NO: 22 targeting exon 7, shows 100% exon skipping at the 50 nM dose.

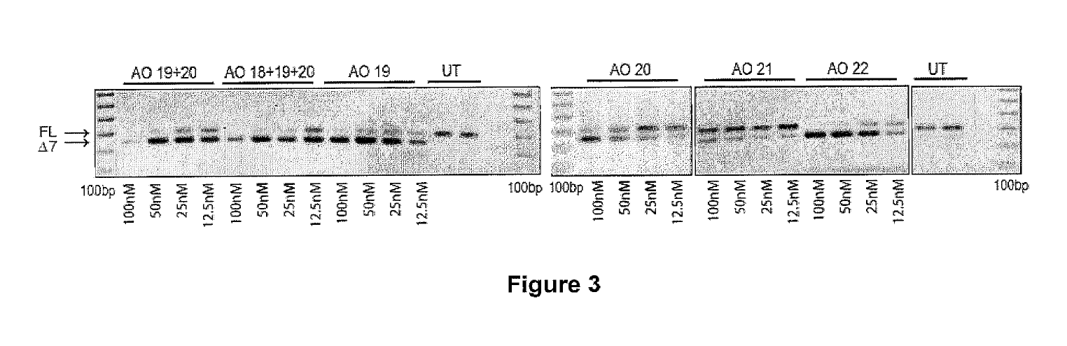

FIG. 4 shows Western blotting data indicating a 75% knockdown of the SMN protein in normal cells following treatment with exon skipping AOs targeting closely around exon 7. AOs were tested at 600 and 300 nM. Levels of SMN expression are normalised against β-tubulin, and treatment groups compared to expression shown by unrelated control AOs. An additional healthy control was used for comparison, as well as an SMA patient sample.

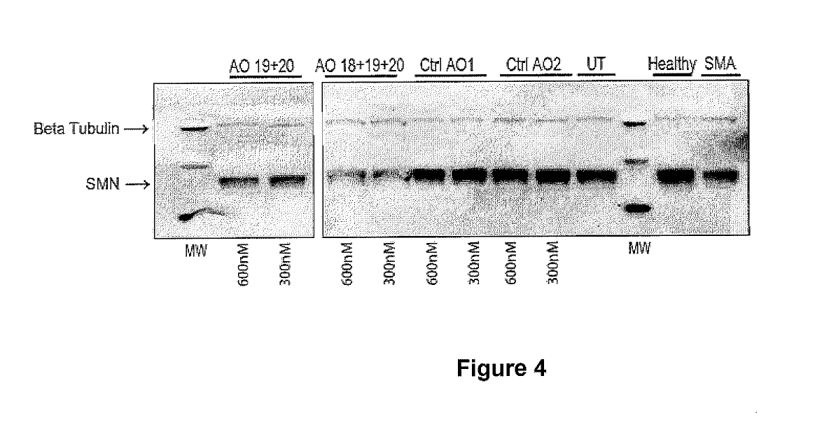

FIG. 5 shows RT-PCR analysis of SMN expression in mouse fibroblasts treated with mouse specific oligomers anticipated to induce exon 7 skipping. This figure shows mouse SMN exon 7 skipping after the exon 7 donor site was targeted with AO SEQ ID NOs: 23-26, specific to the mouse donor splice site. These AOs are different from human specific AO SEQ ID NOs: 18-22, as the human and mouse genes are divergent in this region, but annealed around the same region. Mouse AO SEQ ID NOs: 23 and 24 (target the same co-ordinates as human AO SEQ ID NOs: 19 and 20), show limited skipping at 600 and 300 nM, inducing high levels of cell death. However, AO SEQ ID NO: 26, shifting 6 bases closer to the exon than the human AO SEQ ID NO: 22, shows greater than 50% exon skipping at the low dose of 50 nM, inducing a FL/Δ7 ratio similar to that seen in SMA patient fibroblasts.

FIG. 6 shows RT-PCR studies indicating changes in the amounts of SMN-FL and SMNΔ7 RNA (indicated with arrow) in SMA patient fibroblasts after transfection with oligomers directed to intron 6 (AO SEQ ID NOs: 29-41) at 25, 50 and 100 nM. AOs show a clear close response targeting regions 120 to 250 bases upstream of exon 7. AO SEQ ID 29 targeting 250 bases upstream of exon 7 induces almost 90% exon inclusion at 100 nM.

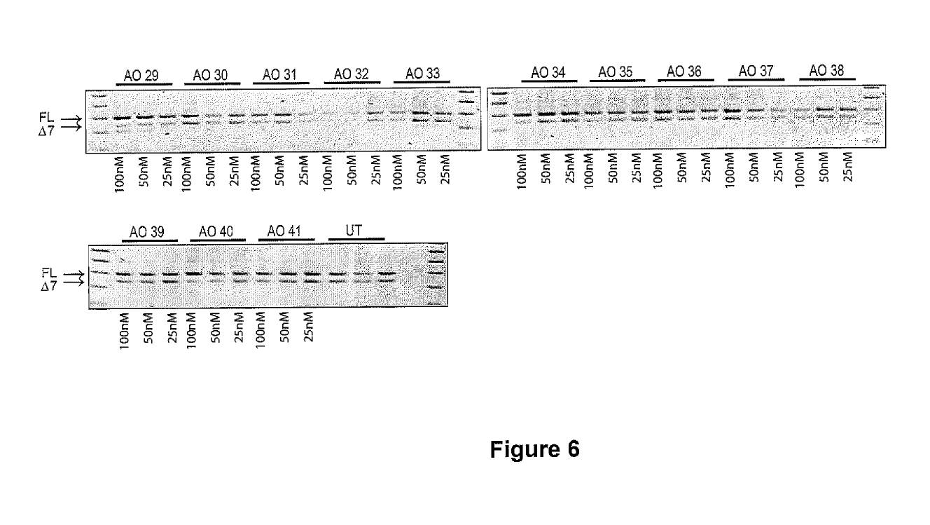

FIG. 7 shows RT-PCR studies indicating changes in the amounts of SMN-FL and SMNΔ7 RNA (indicated with arrow) in SMA patient fibroblasts after transfection with oligomers directed to intron 7 (AO SEQ ID NOs: 7-13, 42, 43, 46-55) at 25, 50 and 100 nM. AO SEQ ID Nos 53 and 54 target 250 bases downstream of exon 7 and promote almost 90% exon retention at 100 nM.

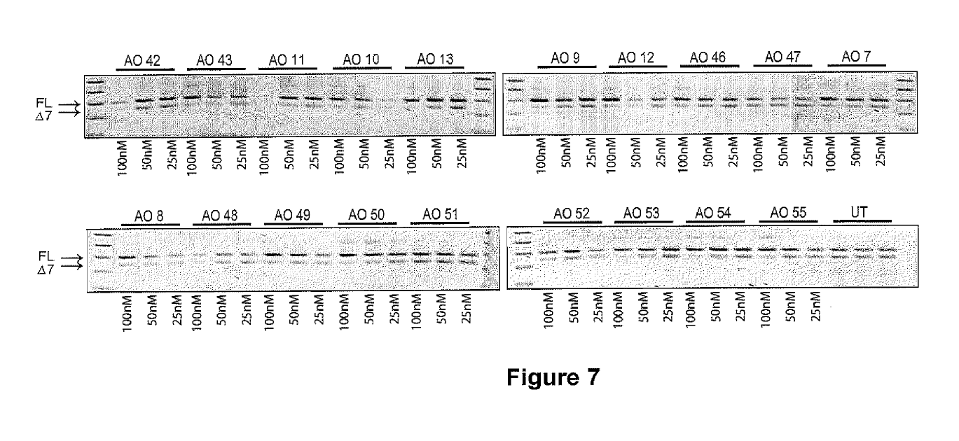

FIG. 8 shows RT-PCR studies indicating changes in the amounts of SMN-FL and SMNΔ7 RNA (indicated with arrow) in SMA patient fibroblasts after transfection with oligomers directed to intron 7 (AO SEQ ID NOs: 10, 44 and 45) at 25, 50 and 100 nM. SEQ ID NOs 44 and 45 are 28 and 30 bases and have greater GC content that AO SEQ ID 10, improving exon 7 retention.

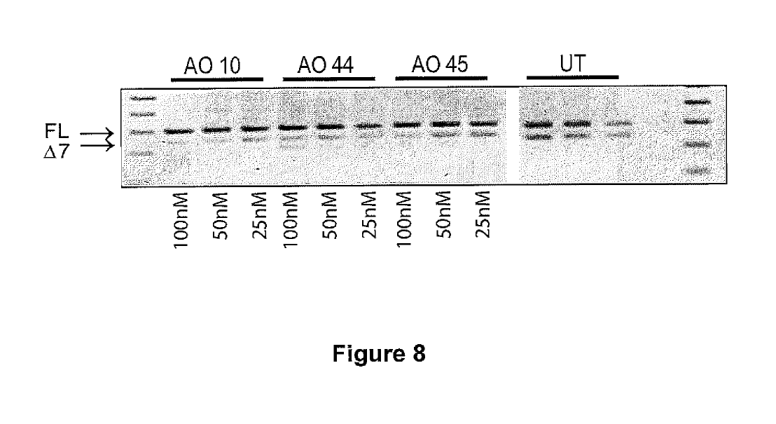

FIG. 9 shows RT-PCR studies indicating changes in the amounts of SMN-FL and SMNΔ7 RNA (indicated with arrow) in SMA patient fibroblasts after transfection with oligomers directed to intron 7 (AO SEQ ID NOs: 10 and 56) at 50, 100 and 200 nM. AO SEQ ID 56 binds intron 7 at two points either side of SEQ ID NO 10 to produce a stapling AO, improving exon 7 retention.

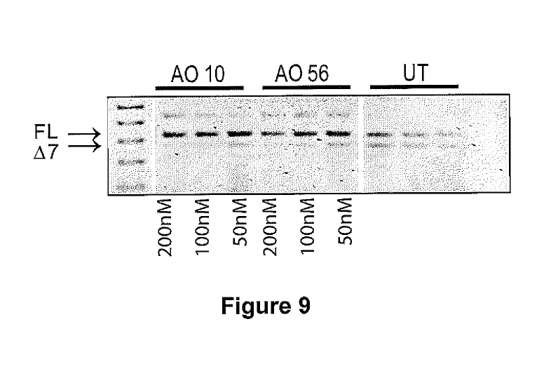

FIG. 10 shows RT-PCR studies indicating changes in the amounts of SMN-FL, SMNΔ7 RNA and SMN-int/ex7 included transcripts (indicated with arrow) in SMA patient fibroblasts after transfection by electroporation with oligomers directed to exon 8 (AO SEQ ID NOs: 14-17) at 250 and 500 nM. All AOs show almost 100% inclusion of the exon and intron 7.

FIG. 11 is a schematic of the binding locations of the AOs of the present invention in the SMN gene. The efficiency of the AO at inducing exon 7 inclusion is represented by “+” according to the scale.

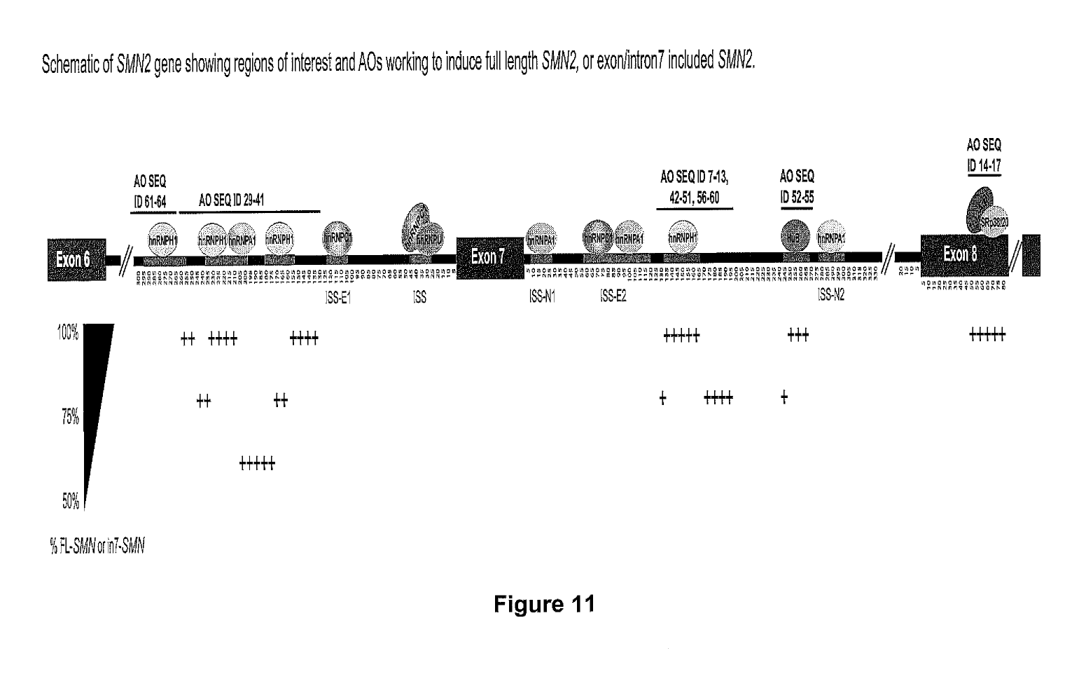

### DETAILED DESCRIPTION OF THE INVENTION

### Antisense Oligomers

According to a first aspect of the invention, there is provided antisense oligomers capable of binding to a selected target on an SMN gene transcript to modify pre-mRNA splicing in an SMN gene transcript or part thereof. Broadly, there is provided an isolated or purified antisense oligomer for inducing exon inclusion in an SMN gene transcript or part thereof.

By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An antisense oligonucleotide can be designed to block or inhibit or modulate translation of mRNA or to inhibit or modulate pre-mRNA splice processing, or induce degradation of targeted mRNAs, and may be said to be “directed to” or “targeted against” a target sequence with which it hybridizes. In certain embodiments, the target sequence includes a region including a 3′ or 5 splice site of a pre-processed mRNA, a branch point, or other sequence involved in the regulation of splicing. The target sequence may be within an exon or within an intron or spanning an intron/exon junction.

In certain embodiments, the antisense oligonucleotide has sufficient sequence complementarity to a target RNA (i.e., the RNA for which splice site selection is modulated) to block a region of a target RNA (e.g., pre-mRNA) in an effective manner. In exemplary embodiments, such blocking of SMN pre-mRNA serves to modulate splicing, either by masking a binding site for a native protein that would otherwise modulate splicing and/or by altering the structure of the targeted RNA. In some embodiments, the target RNA is target pre-mRNA (e.g., SMN gene pre-mRNA).

An antisense oligonucleotide having a sufficient sequence complementarity to a target RNA sequence to modulate splicing of the target RNA means that the antisense agent has a sequence sufficient to trigger the masking of a binding site for a native protein that would otherwise modulate splicing and/or alters the three-dimensional structure of the targeted RNA. Likewise, an oligonucleotide reagent having a sufficient sequence complementary to a target RNA sequence to modulate splicing of the target RNA means that the oligonucleotide reagent has a sequence sufficient to trigger the masking of a binding site for a native protein that would otherwise modulate splicing and/or alters the three-dimensional structure of the targeted RNA.

Selected antisense oligonucleotides can be made shorter, e.g., about 12 bases, or longer, e.g., about 40 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the antisense oligomer is selected from the group comprising the sequences set forth in Tables 1, 2, 5 and 6, bar SEQ ID NOs 1 to 6

In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence. In certain embodiments, a minimum length of complementary bases may be required to achieve the requisite binding Tm, as discussed herein.

In certain embodiments, oligonucleotides as long as 40 bases may be suitable, where at least a minimum number of bases, e.g., 10-12 bases, are complementary to the target sequence. In general, however, facilitated or active uptake in cells is optimized at oligonucleotide lengths of less than about 30 bases. For PMO oligonucleotides, described further herein, an optimum balance of binding stability and uptake generally occurs at lengths of 18-25 bases. Included are antisense oligonucleotides (e.g., PMOs, PMO-X, PNAs, LNAs, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 bases.

In certain embodiments, antisense oligonucleotides may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones which are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an antisense oligonucleotide is not necessarily 100% complementary to the target sequence, it is effective to stably and specifically bind to the target sequence, such that splicing of the target pre-RNA is modulated.

The stability of the duplex formed between an oligonucleotide and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, antisense oligonucleotides may have a binding Tm, with respect to a complementary-sequence RNA, of greater than body temperature and preferably greater than about 45° C. or 50° C. Tm's in the range 60-80° C. or greater are also included.

Additional examples of variants include oligonucleotides having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS:7 to 17 and 29 to 64.

More specifically, there is provided an antisense oligomer capable of binding to a selected target site to modify pre-mRNA splicing in an SMN gene transcript or part thereof. Preferably, the antisense oligomer is capable of binding to a selected target site to induce exon 7 inclusion in an SMN gene transcript. The antisense oligomer is preferably selected from those provided in Tables 1, 2 and 5 to 7 bar SEQ ID NOs 1 to 6.

The SMN gene may be SMN2. As such, the antisense oligomer may be a molecule directed towards a target sequence in intron 6, intron 7, or exon 8 of SMN2 pre-mRNA. The target site may be a silencer site in the SMN2 gene transcript.

The antisense oligomer may be an antisense oligomer capable of inducing exon 7 inclusion and, if desired, intron 7 inclusion in mature SMN2 mRNA. As such, the antisense oligomer of the invention may be used to generate SMN2 transcripts containing exon 7 and intron 7, wherein exon 7 contains the normal termination codon, and intron 7 becomes part of the 3′ UTR of the SMN gene transcript. Preferably, such antisense oligomers are chosen from those in Table 2.

Advantageously, the invention also provides a method for enhancing or modulating SMN2 intron 7 and exon 7 inclusion in a transcript, the method including the step of using one or more antisense oligomers of the invention.

The antisense oligomer of the invention may be selected to be an antisense oligomer capable of binding to a selected SMN target site, wherein the target site is an mRNA splicing site selected from a splice donor site, splice acceptor sites, or exonic splicing elements.

There is also provided a combination or “cocktail” of two or more antisense oligomers capable of binding to a selected target to induce exon inclusion. Alternatively, exon inclusion may be induced by two or more antisense oligomers joined together or a construct comprising two or more oligomers.

The invention further provides a method for manipulating splicing in an SMN gene transcript, the method including the step of:
a) providing one or more of the antisense oligomers as described herein and allowing the oligomer(s) to bind to a target nucleic acid site.

According to yet another aspect of the invention, there is provided a splice manipulation target nucleic acid sequence for SMN comprising the DNA or cDNA equivalents of the nucleic acid sequences set forth in Tables 1, 2 and 5 to 7 bar SEQ ID NOs 1 to 6. The sequences are preferably selected from the group consisting of any one or more of SEQ ID NOs: 7 to 17 and 29 to 64, specifically SEQ ID NOs: 7 to 13, 29, 44, 53 and 54, more specifically SEQ ID NOs: 7 to 13, most specifically SEQ ID NO. 10, and sequences complementary thereto.

Designing antisense oligomers to completely mask consensus splice sites may not necessarily generate a change in splicing of the targeted exon. Furthermore, the inventors have discovered that size or length of the antisense oligomer itself is not always a primary factor when designing antisense oligomers. With some targets such as SMN2 exon 7, antisense oligomers as short as 15 bases were able to induce some exon inclusion, in certain cases more efficiently than other longer (20-30 bases) oligomers directed to the same exon.

The inventors have also discovered that there does not appear to be any standard motif that can be blocked or masked by antisense oligomers to redirect splicing. Preferably, the present disclosure aims to provide antisense oligomers capable of binding to a selected target in the SMN, preferably the SMN2, pre-mRNA to induce efficient and consistent inclusion of exon 7 as well as, in one embodiment, intron 7. As SMA and other SMN-related pathologies arise from mutations that preclude the synthesis of a functional SMN gene product, splicing branch points and exon recognition sequences or splice enhancers are target sites for modulation of mRNA splicing.

More specifically, the antisense oligomer may be selected from those set forth in Tables 1, 2 and 5 to 7 bar SEQ ID NOs 1 to 6. The sequences are preferably selected from the group consisting of any one or more of any one or more of SEQ ID NOs:7-17 and 29-64, specifically SEQ ID NOs: 7 to 13, 29, 44, 53 and 54, more specifically SEQ ID NOs: 7 to 13, most specifically SEQ ID NO. 10, and combinations or cocktails thereof. This includes sequences which can hybridise to such sequences under stringent hybridisation conditions, sequences complementary thereto, sequences containing modified bases, modified backbones, and functional truncations or extensions thereof which possess or modulate pre-mRNA processing activity in an SMN gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an antisense oligomer need not be 100% complementary to that of its target sequence to be specifically hybridisable. An antisense oligomer is specifically hybridisable when binding of the compound to the target DNA or RNA molecule interferes with the normal function of the target DNA or RNA product, and there is a sufficient degree of complementarity to avoid non-specific binding of the antisense oligomer to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of antisense oligomers may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the antisense oligonucleotide to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the polynucleotide sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an antisense oligomer of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the antisense oligomers set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The oligomers of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

It will also be appreciated that there may be conditions or individuals in which it is required to modulate the level of exon inclusion, and this may be achievable by selecting a specific oligomer or combinations thereof which will induce the desired level of exon 7 or exon7+intron 7 inclusion.

The term “modulate” includes to “increase” or “decrease” one or more quantifiable parameters, optionally by a defined and/or statistically significant amount. By “increase” or “increasing,” “enhance” or “enhancing,” or “stimulate” or “stimulating,” refers generally to the ability of one or antisense compounds or compositions to produce or cause a greater physiological response (i.e., downstream effects) in a cell or a subject relative to the response caused by either no antisense compound or a control compound. Relevant physiological or cellular responses (in vivo or in vitro) will be apparent to persons skilled in the art, and may include increases in the inclusion of exon 7 in a SMN-coding pre-mRNA, or increases in the expression of functional SMN enzyme in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) the amount produced by no antisense compound (the absence of an agent) or a control compound. The term “reduce” or “inhibit” may relate generally to the ability of one or more antisense compounds or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a glycogen storage disease such as Pompe disease, for example, a decrease in the accumulation of glycogen in one or more tissues. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an antisense oligomer may vary, as long as it is capable of binding selectively to the intended location within the pre-mRNA molecule. The length of such sequences can be determined in accordance with selection procedures described herein. Generally, the antisense oligomer will be from about 10 nucleotides in length, up to about 50 nucleotides in length. It will be appreciated, however, that any length of nucleotides within this range may be used in the method. Preferably, the length of the antisense oligomer is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “antisense oligonucleotide” or “oligonucleotide” refers to a linear sequence of nucleotides, or nucleotide analogs, that allows the nucleobase to hybridize to a target sequence in an RNA by Watson-Crick base pairing, to form an oligonucleotide:RNA heteroduplex within the target sequence. The terms “antisense oligonucleotide”, “antisense oligomer”, “oligomer” and “compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, among other antisense agents known in the art.

Included are non-naturally-occurring oligonucleotides, or “oligonucleotide analogs”, including oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing antisense oligomers is the methylation of the 2 hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-mRNA during duplex formation with the antisense oligomers, the antisense oligomers used in the method may be adapted to minimise or prevent cleavage by endogenous RNase H. This property is highly preferred, as the treatment of the RNA with the unmethylated oligomers, either intracellular or in crude extracts that contain RNase H, leads to degradation of the pre-mRNA:antisense oligomer duplexes. Any form of modified antisense oligomers that is capable of by-passing or not inducing such degradation may be used in the present method. The nuclease resistance may be achieved by modifying the antisense oligomers of the invention so that it comprises partially unsaturated aliphatic hydrocarbon chain and one or more polar or charged groups including carboxylic acid groups, ester groups, and alcohol groups.

An example of antisense oligomers which when duplexed with RNA are not cleaved by cellular RNase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant antisense oligomers of the invention may have at least one of the last 3′-terminus nucleotides fluoridated. Still alternatively, the nuclease resistant antisense oligomers of the invention have phosphorothioate bonds linking between at least two of the last 3-terminus nucleotide bases, preferably having phosphorothioate bonds linking between the last four 3′-terminal nucleotide bases.

Increased splice-switching may also be achieved with phosphorodiamidate morpholino oligomer (PMO) chemistry. AO-induced splice modification of the human or mouse SMN gene transcripts have generally used either oligoribonucleotides, PNAs, 2OMe or MOE modified bases on a phosphorothioate backbone. Although 2OMeAOs are used for oligo design, due to their efficient uptake in vitro when delivered as cationic lipoplexes, these compounds are susceptible to nuclease degradation and are not considered ideal for in vivo or clinical applications.

Antisense oligomers that do not activate RNase H can be made in accordance with known techniques (see, e.g., U.S. Pat. No. 5,149,797). Such antisense oligomers, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous antisense oligomers that do not activate RNase H are available. For example, such antisense oligomers may be oligomers wherein at least one, or all, of the inter-nucleotide bridging phosphate residues are modified phosphates, such as methyl phosphonates, methyl phosphorothioates, phosphoromorpholidates, phosphoropiperazidates and phosphoramidates. For example, every other one of the internucleotide bridging phosphate residues may be modified as described. In another non-limiting example, such antisense oligomers are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

While antisense oligomers are a preferred form of the antisense oligomers, the present invention includes other oligomeric antisense oligomers, including but not limited to oligomer mimetics such as are described below.

Specific examples of preferred antisense oligomers useful in this invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be oligonucleosides.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNaseH activity when bound to a RNA strand and have been shown to exert sustained splice modulation after in vivo administration. (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications.

Another modification of the oligomers of the invention involves chemically linking to the oligomer one or more moieties or conjugates that enhance the activity, cellular distribution or cellular uptake of the oligomer. Such moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Cell penetrating peptides have been added to phosphorodiamidate morpholino oligomers to enhance cellular uptake and nuclear localization. Different peptide tags have been shown to influence efficiency of uptake and target tissue specificity, as shown in Jearawiriyapaisarn et al. (2008), Mol. Ther. 16 9, 1624-1629.

It is not necessary for all positions in a given compound to be uniformly modified, and in fact more than one of the aforementioned modifications may be incorporated in a single compound or even at a single nucleoside within an oligomer. The present invention also includes antisense oligomers that are chimeric compounds. “Chimeric” antisense oligomers or “chimeras,” in the context of this invention, are antisense oligomers, particularly oligomers, which contain two or more chemically distinct regions, each made up of at least one monomer unit, i.e., a nucleotide in the case of an oligomer compound. These oligomers typically contain at least one region wherein the oligomer is modified so as to confer upon the oligomer or antisense oligomer increased resistance to nuclease degradation, increased cellular uptake, and an additional region for increased binding affinity for the target nucleic acid.

The activity of antisense oligonucleotides and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (La, a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

### Methods of Manufacturing Antisense Oligomers

The antisense oligomers used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The antisense oligomers of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

### Therapeutic Agents

The present invention also can be used as a prophylactic or therapeutic, which may be utilised for the purpose of treatment of a genetic disease. Accordingly, in one embodiment the present invention provides antisense oligomers that bind to a selected target in the SMN2 pre-mRNA to induce efficient and consistent exon 7 inclusion described herein, in a therapeutically effective amount, admixed with a pharmaceutically acceptable carrier, diluent, or excipient.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, Pa., (1990).

In a more specific form of the invention there are provided pharmaceutical compositions comprising therapeutically effective amounts of one or more antisense oligomers of the invention together with pharmaceutically acceptable diluents, preservatives, solubilizers, emulsifiers, adjuvants, and/or carriers. Such compositions include diluents of various buffer content (e.g. Tris-HCl, acetate, phosphate), pH and ionic strength and additives such as detergents and solubilizing agents (e.g. Tween 80, Polysorbate 80), anti-oxidants (e.g., ascorbic acid, sodium metabisulfite), preservatives (e.g. Thimersol, benzyl alcohol) and bulking substances (e.g., lactose, mannitol). The material may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, Pa. 18042) pages 1435-1712 that are herein incorporated by reference. The compositions may be prepared in liquid form, or may be in dried powder, such as a lyophilised form.

It will be appreciated that pharmaceutical compositions provided according to the present invention may be administered by any means known in the art. Preferably, the pharmaceutical compositions for administration are administered by injection, orally, topically or by the pulmonary, or nasal route. The antisense oligomers are more preferably delivered by intravenous, intra-arterial, intraperitoneal, intramuscular, or subcutaneous routes of administration. The appropriate route may be determined by one of skill in the art, as appropriate to the condition of the subject under treatment. Vascular or extravascular circulation, the blood or lymph system, and the cerebrospinal fluid are some non-limiting sites where the antisense oligomer may be introduced. Direct CNS delivery may be employed, for instance, intracerebral ventribular or intrathecal administration may be used as routes of administration.

### Antisense Oligomer Based Therapy

Also addressed by the present invention is the use of antisense oligomers of the present invention, for manufacture of a medicament for modulation of a genetic disease.

Therefore, according to a still further aspect of the invention, there is provided one or more antisense oligomers as described herein for use in an antisense oligomer-based therapy.

According to another aspect of the invention, there is provided a method of treating an SMN-related pathology in a patient, comprising the step of:
a) administering to the patient a composition comprising one or more antisense oligomers as described herein.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The invention further provides a pharmaceutical, prophylactic, or therapeutic composition for the treatment of an SMN-related pathology in a patient, the composition comprising:
a) one or more antisense oligomers as described herein, and
b) one or more pharmaceutically acceptable carriers and/or diluents.

The composition may comprise about 1 nM to 1000 nM of each of the desired antisense oligomer(s) of the invention. Preferably, the composition may comprise about 1 nM to 500 nM, 50 nM to 750 nM, 10 nM to 500 nM, 1 nM to 100 nM, 1 nM to 50 nM, 1 nM to 40 nM, 1 nM to 30 nM, 1 nM to 20 nM, most preferably between 1 nM and 10 nM of each of the antisense oligomer(s) of the invention.

According to another aspect of the invention there is provided the use of one or more antisense oligomers as described herein in the manufacture of a medicament for the modulation or control of SMN-related pathologies.

The SMN-related pathology may be a muscular atrophy, such as Spinal Muscular Atrophy (SMA) arising from loss of a functional SMN gene product.

The present invention further provides one or more antisense oligomers adapted to aid in the prophylactic or therapeutic treatment of a genetic disorder such as an SMN-related pathology in a form suitable for delivery to a patient.

According to another aspect, the invention provides a method for treating a patient suffering from a genetic disease or pathology wherein there is a deleterious mutation in an SMN gene and the effect of the mutation can be abrogated by splice manipulation, comprising the steps of:
a) selecting one or more antisense oligomers as described herein; and
b) administering the antisense oligomers to the patient.

The patient may be a mammal, including a human.

The invention also provides for the use of purified and isolated antisense oligomers as described herein, for the manufacture of a medicament for treatment of an SMN-related genetic disease.

The invention further provides a method of treating a condition characterised by incorrect SMN expression in a patient, particularly a condition associated with SMA, comprising the step of:
a) administering to the patient an effective amount of one or more antisense oligomers as described herein

Preferably, the antisense oligomers administered are relevant to the particular genetic lesion in that patient that led to the incorrect SMN expression in the patient.

Furthermore, the invention provides a method for prophylactically treating a patient to prevent or at least minimise SMA, comprising the step of:
a) administering to the patient an effective amount of one or more antisense oligomers or pharmaceutical composition comprising one or more antisense oligomers.

According to a still further aspect of the invention, there is provided a method of treating a patient with a pathology caused by incorrect, incomplete, or defective SMN-splicing, the method including the step of:
a) administering to the patient an effective amount of one or more antisense oligomers as described herein or a composition as described herein.

The delivery of a therapeutically useful amount of antisense oligomers may be achieved by methods previously published. For example, intracellular delivery of the antisense oligomer may be via a composition comprising an admixture of the antisense oligomer and an effective amount of a block copolymer. An example of this method is described in US patent application US20040248833. Other methods of delivery of antisense oligomers to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

In certain embodiments, the antisense oligonucleotides of the invention can be delivered by transdermal methods (e.g., via incorporation of the antisense oligonucleotides into, e.g., emulsions, with such antisense oligonucleotides optionally packaged into liposomes). Such transdermal and emulsion/liposome-mediated methods of delivery are described for delivery of antisense oligonucleotides in the art, e.g., in U.S. Pat. No. 6,965,025, the contents of which are incorporated in their entirety by reference herein.

It may be desirable to deliver the antisense oligomer in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., Trends Biochem. Sci. 6:77, 1981).

In order for a liposome to be an efficient gene transfer vehicle, the following characteristics should be present: (1) encapsulation of the antisense oligomer of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et al., Biotechniques, 6:682, 1988). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations.

The antisense oligonucleotides described herein may also be delivered via an implantable device. Design of such a device is an art-recognized process, with, e.g., synthetic implant design described in, e.g., U.S. Pat. No. 6,969,400, the contents of which are incorporated in their entirety by reference herein.

Antisense oligonucleotides can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, antisense oligonucleotides may be delivered using, e.g., methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The antisense oligomer may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244:1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68:143-155; Rosenfeld, et al. (1991) Science, 252:431-434); or delivery of a transgene linked to a heterologous promoter-enhancer element via liposomes (Friedmann (1989), supra; Brigham, et al. (1989) Am. J. Med. Sci., 298:278-281; Nabel, et al. (1990) Science, 249:1285-1288; Hazinski, et al. (1991) Am. J. Resp. Cell Molec. Biol., 4:206-209; and Wang and Huang (1987) Proc. Natl. Acad. Sci. (USA), 84:7851-7855); coupled to ligand-specific, cation-based transport systems (Wu and Wu (1988) J. Biol. Chem., 263:14621-14624) or the use of naked DNA, expression vectors (Nabel et al. (1990), supra); Wolff et al. (1990) Science, 247:1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group (Am. J. Med. Sci. (1989) 298:278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, Science (1992) 256:808-813; Barteau et al. (2008), Curr Gene Ther; 8(5):313-23; Mueller et al. (2008). Clin Rev Allergy Immunol; 35(3):164-78; Li et al. (2006) Gene Ther., 13(18):1313-9; Simoes et al. (2005) Expert Opin Drug Deliv; 2(2):237-54.

The antisense oligomers of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e. salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and mucous membranes, as well as rectal delivery), pulmonary, e.g., by inhalation or insufflation of powders or aerosols (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligomers with at least one 2′-O-methoxyethyl modification are believed to be particularly useful for oral administration.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

In one embodiment, the antisense compound is administered in an amount and manner effective to result in a peak blood concentration of at least 200-400 nM antisense oligonucleotide. Typically, one or more doses of antisense oligomer are administered, generally at regular intervals, for a period of about one to two weeks. Preferred doses for oral administration are from about 1 mg to 1000 mg oligomer per 70 kg. In some cases, doses of greater than 1000 mg oligomer/patient may be necessary. For i.v. administration, preferred doses are from about 0.5 mg to 1000 mg oligomer per 70 kg. The antisense oligomer may be administered at regular intervals for a short time period, e.g., daily for two weeks or less. However, in some cases the oligomer is administered intermittently over a longer period of time. Administration may be followed by, or concurrent with, administration of an antibiotic or other therapeutic treatment. The treatment regimen may be adjusted (dose, frequency, route, etc.) as indicated, based on the results of immunoassays, other biochemical tests and physiological examination of the subject under treatment.

An effective in vivo treatment regimen using the antisense oligonucleotides of the invention may vary according to the duration, dose, frequency and route of administration, as well as the condition of the subject under treatment (i.e., prophylactic administration versus administration in response to localized or systemic infection). Accordingly, such in vivo therapy will often require monitoring by tests appropriate to the particular type of disorder under treatment, and corresponding adjustments in the dose or treatment regimen, in order to achieve an optimal therapeutic outcome.

Treatment may be monitored, e.g., by general indicators of disease known in the art. The efficacy of an in vivo administered antisense oligonucleotide of the invention may be determined from biological samples (tissue, blood, urine etc.) taken from a subject prior to, during and subsequent to administration of the antisense oligonucleotide. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

### Kits of the Invention

The invention also provides kits for treatment of a patient with a genetic disease caused by aberrant expression levels of the SMN gene, which kit comprises at least an isolated or purified antisense oligomer for modifying pre-mRNA splicing in an SMN gene transcript or part thereof, packaged in a suitable container, together with instructions for its use.

In a preferred embodiment, the kits will contain at least one antisense oligomer as shown in Tables 1, 2 and 5 to 7, bar SEQ ID NO:s 1 to 6, or a cocktail of antisense oligomers, as described herein. The kits may also contain peripheral reagents such as buffers, stabilizers, etc. there is therefore provided a kit for treatment of a patient with a genetic disease which kit comprises at least an antisense oligomer chosen from Tables 1, 2 and 5 to 7, bar SEQ ID NO:s 1 to 6 and combinations or cocktails thereof, packaged in a suitable container, together with instructions for its use

There is also provided a kit for treatment of a patient with a genetic disease which kit comprises at least an antisense oligomer selected from the group consisting of any one or more of SEQ ID NOs: 7 to 17 and 29 to 64, specifically SEQ ID NOs: 7 to 13, 29, 44, 53 and 54, more specifically SEQ ID NOs: 7 to 13, most specifically SEQ ID NO. 10, and combinations or cocktails thereof, packaged in a suitable container, together with instructions for its use

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

Those of ordinary skill in the field should appreciate that applications of the above method has wide application for identifying antisense oligomers suitable for use in the treatment of many other diseases.

### SMN Genes and SMA

Spinal muscular atrophy (SMA) arises most commonly through genomic deletions of the Survival Motor Neuron 1 (SMN1) gene on chromosome 5, resulting in a deficiency of functional SMN protein. Humans have one or more additional copies of the SMN gene, but the centromeric SMN2 gene copy, or copies, cannot compensate for the loss of SMN1, due to non-productive alternative splicing.

As discussed above, the choice of target selection plays a crucial role in the efficiency of exon inclusion and hence its subsequent application as a potential therapy. Simply designing antisense oligomers to target regions of pre-mRNA presumed to be involved in splicing is no guarantee of efficiently redirecting pre-mRNA splicing. However, optimal target site, oligomer length and chemistry are crucial for efficient splice-switching oligomers. Targets for splicing intervention investigated include the donor and acceptor splice sites, although there are less defined or conserved motifs including exonic splicing enhancers, silencing elements and branch points. The acceptor and donor splice sites have consensus sequences of about 16 and 8 bases respectively. In the present invention, silencer sequences are targeted to thereby promote inclusion of exon 7 into the mature SMN2 gene transcript. The present invention has identified a number of AOs that provide substantial and consistent increases in splice-switching of SMN RNA.

Intranuclear oligomer delivery is also a major challenge. Different cell-penetrating peptides (CPP) localize PMOs to varying degrees in different conditions and cell lines, and novel CPPs have been evaluated by the inventors for their ability to deliver PMOs to the target cells. The terms CPP or “a peptide moiety which enhances cellular uptake” are used interchangeably and refer to cationic cell penetrating peptides, also called “transport peptides”, “carrier peptides”, or “peptide transduction domains”. The peptides, as shown herein, have the capability of inducing cell penetration within about or at least about 30%, 40%, 50%, 60%, 70%, 80%, 90%, or 100% of cells of a given cell culture population and allow macromolecular translocation within multiple tissues in vivo upon systemic administration. CPPs are well-known in the art and are disclosed, for example in U.S. Application No. 201010016215, which is incorporated by reference in its entirety.

This disclosure provides AO-induced splice-switching of the SMN gene transcript, clinically relevant oligomer chemistries, delivery systems, and relevant animal models to direct SMN2 splice manipulation to therapeutic levels. Substantial increases in the amount of full length SMN2 mRNA, and hence SMN protein from SMN2 gene transcription, are achieved by:
1) oligomer refinement in vitro using SMA patient fibroblasts, through experimental assessment of (i) intronic silencing target motifs, (ii) AO length and development of oligomer cocktails, (iii) choice of chemistry, and (iv) the addition of cell-penetrating peptides (CPP) to enhance oligomer delivery.
2) detailed evaluation of a novel approach to generate SMN2 transcripts containing exon 7 and, if desired or required, intron 7 (exon 7 contains the normal termination codon and intron 7 becomes part of the 3′ UTR of the SMN2 gene transcript).
3) validation of splice manipulation therapies in vivo using AO treated mild, moderate and severely affected mouse models of SMA.

As such, it is demonstrated herein that processing of SMN pre-mRNA, particularly SMN2 mRNA, can be manipulated with specific antisense oligomers. In this way functionally significant amounts of SMN protein can be synthesized from the SMN2 gene, thereby reducing the severe pathology associated with muscular atrophies such as, for example, spinal muscular atrophy (SMA).

There is provided the use of one or more antisense oligomers as described herein in the manufacture of a medicament for the modulation or control of a muscular atrophy, such as Spinal Muscular Atrophy (SMA) arising from loss of a functional SMN gene product.

The invention further provides a method of treating a condition associated with SMA characterised by incorrect SMN expression in a patient, comprising the step of:
a) administering to the patient an effective amount of one or more antisense oligomers as described herein.

Preferably, the antisense oligomers administered are relevant to the particular genetic lesion in that patient that led to the incorrect SMN expression in the patient.

Furthermore, the invention provides a method for prophylactically treating a patient to prevent or at least minimise SMA, comprising the step of:
a) administering to the patient an effective amount of one or more antisense oligomers or pharmaceutical composition comprising one or more antisense oligomers.

The antisense oligomer used in the methods of treatment and manufacture of medicaments described are preferably selected from the group comprising the sequences set forth in Tables 1, 2 and 5 to 7 bar SEQ ID NOs 1 to 6. More specifically, the antisense oligomer may be selected from the group consisting of any one or more of SEQ ID NOs: 7 to 17 and 29 to 64, specifically SEQ ID NOs: 7 to 13, 29, 44, 53 and 54, more specifically SEQ ID NOs: 7 to 13, most specifically SEQ ID NO. 10, and combinations or cocktails thereof.

### Combination Therapies

The AOs of the present invention may also be used in conjunction with alternative therapies, such as drug therapies.

High throughput drug screens have identified possible compounds (Andreassi, 2001 #19; Brichta, 2003 #256; Chang, 2001 #257; Kernochan, 2005 #255; Sumner, 2003 #14) for use in SMA treatment, but further extensive experimentation is needed for potential application of these compounds. These compounds do not need to exert an effect during SMN gene transcript splicing. Up-regulating SMN expression or stabilizing the SMN transcripts could be potentially therapeutic, and it is likely that different mechanisms may be harnessed.

A further screening study of over 550,000 compounds has been conducted, from which 17 distinct compounds were confirmed as increasing SMN expression. One of these compounds, a C5-substituted quinazoline, appears to exert its effect on SMN expression through a mechanism different to that induced by AOs. Given the different mechanisms of action of compounds identified as compared to the AOs of the present invention, therapy with a combination of agents may increase efficacy.

### General

Those skilled in the art will appreciate that the invention described herein is susceptible to variations and modifications other than those specifically described. It is to be understood that the invention includes all such variation and modifications. The invention also includes all of the steps, features, compositions and compounds referred to or indicated in the specification, individually or collectively and any and all combinations or any two or more of the steps or features.

The present invention is not to be limited in scope by the specific embodiments described herein, which are intended for the purpose of exemplification only. Functionally equivalent products, compositions and methods are clearly within the scope of the invention as described herein.

Sequence identity numbers (“SEQ ID NO:”) containing nucleotide and amino acid sequence information included in this specification are collected at the end of the description and have been prepared using the program PatentIn Version 3.0. Each nucleotide or amino acid sequence is identified in the sequence listing by the numeric indicator <210> followed by the sequence identifier (e.g. <210>1, <210>2, etc.). The length, type of sequence and source organism for each nucleotide or amino acid sequence are indicated by information provided in the numeric indicator fields <211>, <212> and <213>, respectively. Nucleotide and amino acid sequences referred to in the specification are defined by the information provided in numeric indicator field <400> followed by the sequence identifier (e.g. <400>1, <400>2, etc.).

An antisense oligomer nomenclature system was proposed and published to distinguish between the different antisense oligomers (see Mann et al., (2002) J Gen Med 4, 644-654). This nomenclature became especially relevant when testing several slightly different antisense oligomers, all directed at the same target region, as shown below:

### H # A/D (x:y).

The first letter designates the species (e.g. H: human, M: murine)
“#” designates target exon number.
“A/D” indicates acceptor or donor splice site at the beginning and end of the exon, respectively.
(x y) represents the annealing coordinates where “−” or “+” indicate intronic or exonic sequences respectively. As an example, A(−6+18) would indicate the last 6 bases of the intron preceding the target exon and the first 18 bases of the target exon. The closest splice site would be the acceptor so these coordinates would be preceded with an “A”. Describing annealing coordinates at the donor splice site could be D(+2−18) where the last 2 exonic bases and the first 18 intronic bases correspond to the annealing site of the antisense oligomer. Entirely exonic annealing coordinates that would be represented by A(+65+85), that is the site between the 65th and 85th nucleotide, inclusive, from the start of that exon.

The entire disclosures of all publications (including patents, patent applications, journal articles, laboratory manuals, books, or other documents) cited herein are hereby incorporated by reference. No admission is made that any of the references constitute prior art or are part of the common general knowledge of those working in the field to which this invention relates.

As used herein the term “derived” and “derived from” shall be taken to indicate that a specific integer may be obtained from a particular source albeit not necessarily directly from that source.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

When antisense oligomer(s) are targeted to nucleotide sequences involved in positive recognition and subsequent splicing of exons within pre-mRNA sequences, normal splicing of the exon may be inhibited, causing the splicing machinery to by-pass the entire targeted exon from the mature mRNA. Conversely, it is possible to target negative splice control elements in a pre-mRNA, exonic or intronic silencer elements in order to promote incorporation of a particular exon, or region of intronic sequence, into the mature gene transcript. In many genes, deletion of an entire exon would lead to the production of a non-functional protein through the loss of important functional domains or the disruption of the reading frame. In some proteins, however, it is possible to shorten the protein by deleting one or more exons (without disrupting the reading frame) from within the protein without seriously altering the biological activity of the protein. Typically, such proteins have a structural role and/or possess functional domains at their respective ends. However, the present invention describes antisense oligomers capable of binding to specified SMN2 pre-mRNA targets and re-directing processing of that gene so that exon 7, normally omitted from the SMN2 gene transcript, (and if desired, intron 7) is included in the mature mRNA.

### EXAMPLES

This disclosure presents data confirming and identifying several previously unreported motifs that promote exon 7 inclusion in SMN, particularly SMN2, mRNA.

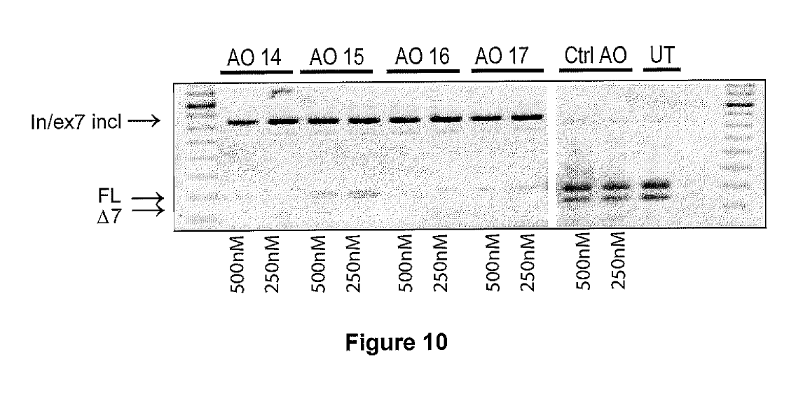

In addition, the inventors induced an SMN2 mRNA retaining both exon 7 and intron 7 (SMN+int7), and demonstrated this effect with four AOs, three of which are overlapping AOs as shown in FIG. 10. This product cannot arise from DNA contamination since the genomic product spanning exons 4-8 is in excess of 10 kb. Since the normal stop codon for SMN is in exon 7, SMN+int7 transcripts should encode a normal protein and intron 7 is included in the 3′ UTR. There is minimal homology between the 3′UTRs of the mouse SMN (355 bases) and the human SMN (444 bases) genes, particularly after the termination codon and flanking the polyadenylation signals.

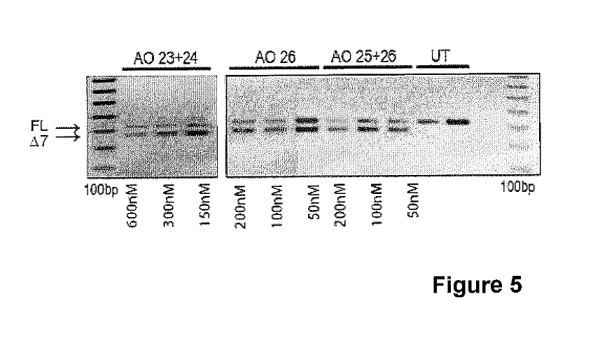

Although targeting the same coordinates in human and mouse exons of equivalent genes may lead to efficient redirection of splicing, the inventors have noted more examples where different splicing patterns were induced. These discrepancies included exon skipping in one species and not the other and induction of distinct splicing patterns, including multiple exon skipping or cryptic splice site activation in, as an example, the dystrophin gene transcript of one species and not the other. Targeting the same region of the mouse SMN exon 7 donor splice site with an oligomer that induced complete skipping of exon 7 in SMA patient fibroblasts led to pronounced but incomplete excision of the exon, as shown in FIG. 5.

It may be argued that exon skipping would not be complete since, unlike humans, the mouse has only one SMN gene that does not carry any polymorphisms weakening exon 7 recognition. The deficiencies of mouse models expressing SMN2 is a recognised limitation, but there is currently no alternative.

### General Methods and Techniques

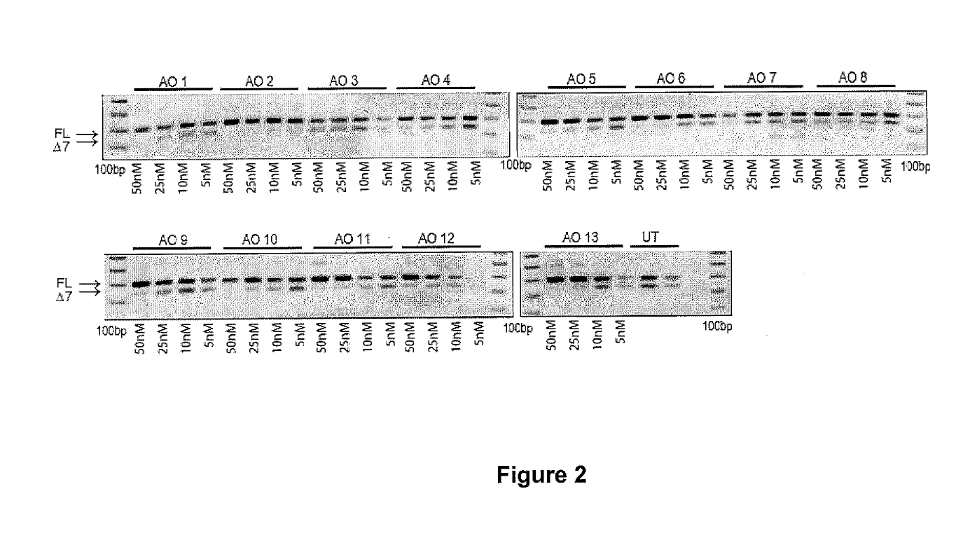

An AO walk across ˜270 bases of introns 6 and 7, immediately flanking exon 7, was undertaken to identify potential silencing motifs. These AOs were transfected into SMA patient fibroblasts and the determination of inclusion of exon 7 in the SMN transcript was undertaken by Reverse Transcriptase-PCR and comparison of the ratios of SMN-FL and SMN Δ7, as shown in FIG. 2.

The retention of intron 7 in the mature SMN2 transcript has several possible consequences, including increased or decreased stability. The inventors used 3′RACE to confirm the normal polyadenylation site is used for SMN-FL, SMN Δ 7 and SMN+int7 (data not shown).

### Cell Propagation and Transfection:

SMA normal or mouse dermal fibroblasts were propagated using well established techniques. SMA I patient cells were seeded and propagated in 75 cm2 tissue culture flasks, transferred to 24 well plates and transfected with 2OMeAO cationic lipoplexes (Lipofectin:oligo ratio of 2:1) over a range of concentrations. For nucleofection, PMOs were transfected into cells via electroporation shock, using a LONZA Nucleofection machine, following manufacturor's instruction. Transfected cells were typically incubated for 48 hours, unless otherwise indicated, before RNA was extracted for analysis using acid phenol extraction (Trizol). RNA samples were treated with RNase free DNAse 1, although minor DNA contamination is not problematic, as intron 6 is in excess of 6 kb.

### Oligomer Nomenclature:

The nomenclature system defines species, exon number, acceptor or donor targeting and annealing coordinates, where “−” indicates intronic position and “+” specifies exonic location from the splice site (Mann, 2002 #114), as described herein. Some detailed oligomer annealing coordinates are shown in Tables 1-6.

### RT-PCR Analysis:

One step RT-PCR using Superscript III: ˜100 ng of total RNA was used as a template and incubated for 30 min at 55° C., and at 94° C. for 2 min, before 25 rounds of 94° C. for 40 sec, 56° C. for 1 min and 68° C. for 1 min using exon 4F and 8R primers. PCR products were fractionated on 2% agarose gels in Tris-Acetate-EDTA buffer and the images captured on a Chemismart-3000 gel documentation system and analysed with Bio1D software to quantitate band weight and estimate ratios of SMN-FL, SMN-int7 and SMN Δ7. Product identity was confirmed by band purification and DNA sequencing as necessary.

### Western Blotting:

Proteins were extracted from treated cultures after three days and prepared according to Cooper et al., 2003, but with 15% SDS. SDS-PAGE electrophoresis was performed using NuPAGE Novex 4-12% BIS/Tris gels run at 200V for 55 mins. Proteins were transferred to Pall Fluorotrans W PVDF membranes at 30 V for 1 hour at 18° C. MANSMA1 antibody was applied at 1:100 dilution overnight at 4° C. and immunodetection used an Invitrogen Western Breeze kit. Quantification was performed on a Vilber Lourmat Chemi-Smart 3000 system using Chemi-Capt software for image acquisition and Bio-1D software for image analysis. β-tubulin was detected by a mouse monoclonal antibody (BD Pharmingen, Cat. no 556321), as a reference loading protein, with loadings normalized compared to the β-tubulin.

### Results

### 1: Improved Oligomer Design to Enhance SMN Exon 7 Inclusion

### Oligo Walking and Refinement Using SMA Fibroblasts:

Introns 6 and 7 were screened for responsive motifs, since it has been found that targeting intra-exon 7 motifs does not lead to efficient induction of the SMN-FL transcript (Hua, 2008). A large number of 2OMeAOs targeted to introns 6 and 7 were designed and evaluated, and several silencing elements identified both upstream (intron 6) and downstream (intron 7) of exon 7.

### Oligomer Backbone Chemistries:

Upon identification of oligomer sequences shown to be most efficient at inducing SMN-FL or SMN+int7 transcripts, new compounds may be prepared using different backbone chemistries. 2′ modified bases on a phosphorothioate backbone (2OMe or phosphorodiamidate morpholino backbone (PMO) may be used, as PMOs generally have a much greater splice switching potential in vivo than the phosphorothioate backbone oligomers.

### Delivery:

Oligomers were efficiently delivered and assessed in vitro after transfection as cationic lipoplexes or by electroporation techniques. Additional techniques that may be used include coupling the oligomers to cell penetrating peptides (CPPs).

TABLE 1
SEQ ID listing of antisense oligomers inducing SMN2 Exon 7 inclusion into transcript

| SEQ ID | | Co- | | Length | % FL SMN |
|---|---|---|---|---|---|
| NO | | ordinates | Sequence | (bases) | at 50 nM |
| 1 | ISS-N1 | SMN2.7D(-10-29) | 5′ AUU CAC UUU CAU AAU GCU GG 3′ | 20 | av. 87 |
| 2 | | SMN2.7D(-10-34) | 5′ GUA AGA UUC ACU UUC AUA AUG CUG G 3′ | 25 | av. 98 |

Intron 6

| | | | | | |
|---|---|---|---|---|---|
| 3 | | SMN2.7A(-70-48) | 5′ GAU AGC UAU AUA UAG AUA GCU UU 3′ | 23 | 84.4 |
| 4 | | SMN2.7A(-70-45) | 5′ AUA GAU AGC UAU AUA UAG AUA GCU UU 3′ | 26 | 97.3 |
| 5 | | SMN2.7A(-67-48) | 5′ GAU AGC UAU AUA UAG AUA GC 3′ | 20 | 89.3 |
| 6 | | SMN2.7A(-58-39) | 5′ AUA GAU AUA GAU AGC UAU AU 3′ | 20 | 99.0 |
| | Intron 7 | | | | |
| 7 | | SMN2.7D(-152-174) | AUU AAC CUU UUA UCU AAU AGU UU | 23 | av. 79.4 |
| 8 | | SMN2.7D(-149-174) | AUU AAC CUU UUA UCU AAU AGU UUU GG | 26 | av. 84.0 |
| 9 | | SMN2.7D(-140-159) | AAU AGU UUU GGC AUC AAA AU | 20 | av. 84.5 |
| 10 | | SMN2.7D(-137-159) | AAU AGU UUU GGC AUC AAA AUU CU | 23 | 97.8 |
| 11 | | SMN2.7D(-134-159) | AAU AGU UUU GGC AUC AAA AUU CUU UA | 26 | 82.0 |
| 12 | | SMN2.7D(-143-162) | UCU AAU AGU UUU GGC AUC AA | 20 | 83.3 |
| 13 | | SMN2.7D(-140-162) | UCU AAU AGU UUU GGC AUC AAA AU | 23 | 94.9 |

TABLE 2
SEQ ID listing of antisense oligomers inducing SMN2 Exon 7 and Intron 7 inclusion
into transcript.

| | | | | |
|---|---|---|---|---|
| SEQ ID | | | | Length |
| NO | Region | Co-ordinates | Sequence | (bases) |

Exon 8

| | | | |
|---|---|---|---|
| 14 | hSMN2.8A(+39+58) | 5′ GAU CUG UCU GAU CGU UUC UU 3′ | 20 |
| 15 | hSMN2.8A(+59+83) | 5′ AUC UUC UAU AAC GCU UCA CAU UCC A 3′ | 25 |
| 16 | hSMN2.8A(+55+79) | 5′ UCU AUA ACG CUU CAC AUU CCA GAU C 3′ | 25 |
| 17 | hSMN2.8A(+57+81) | 5′ CUU CUA UAA CGC UUC ACA UUC CAG A 3′ | 25 |

TABLE 3
SEQ ID listing of antisense oligomers inducing human SMN1 Exon 7 skipping from transcript.

| SEQ ID | | | | Length |
|---|---|---|---|---|
| NO | Region | Co-ordinates | Sequence | (bases) |
| | Intron 6 | | | |
| 18 | | h5MN1.7A(-110-91) | 5′ UUU GUU UCA CAA GAC AUU UU 3 | 20 |
| | Exon 7 | | | |
| 19 | | h5MN1.7A(+7+31) | 5′ ACC UUC CUU CUU UUU GAU UUU GUC U 3′ | 25 |
| 20 | | h5MN1.7D(+17-13) | 5′ CUG GCA GAC UUA CUC CUU AAU UUA AGG AAU 3′ | 30 |
| 21 | | h5MN1.7A(+6+27) | 5′ UCC UUC UUU UUG AUU UUG UCU G 3′ | 22 |
| 22 | | h5MN1.7A(+13+32) | 5′ CAC CUU CCU UCU UUU UGA UU 3′ | 20 |

TABLE 4
SEQ ID listing of antisense oligomers inducing mouse Smn Exon 7 skipping from
transcript.

| SEQ ID | | | | Length |
|---|---|---|---|---|
| NO | | Co-ordinates | Sequence | (bases) |
| | Exon 7 | | | |
| 23 | | mSmn7A(+7+31) | 5′ ACU UUC CUU CUU UUU UAU UUU GUC U 3′ | 25 |
| 24 | | mSmn7D(+17-13) | 5′ AUG ACA GAC UUA CUU CUU AAU UUG UAU GUG 3′ | 30 |
| 25 | | mSmn7D(+11-19) | 5′ UUU AAA AUG ACA GAC UUA CUU CUU AAU UUG 3′ | 30 |
| 26 | | mSmn7A(+7+36) | 5′ UGA GCA CUU UCC UUC UUU UUU AUU UUG UCU 3′ | 30 |

TABLE 5
SEQ ID listing of antisense oligomers inducing SMN2 Exon 7 inclusion into transcript

| SEQ ID | | | | Length | % FL SMN |
|---|---|---|---|---|---|
| NO | | Co-ordinates | Sequence | (bases) | at 100 nM |
| | Intron 6 | | | | |
| 29 | | SMN2.7A(-264-245) | ACA ACU UUG GGA GGC GGA GG | 20 | 87 |
| 30 | | SMN2.7A(-258-239) | AAU CCC ACA ACU UUG GGA GG | 20 | 56 |
| 31 | | SMN2.7A(-252-227) | GCU CAU GCC UAC AAU CCC ACU UCU UU | 26 | 72 |
| 32 | | SMN2.7A(-249-227) | GCU CAU GCC UAC AAU CCC ACU UC | 23 | 63 |
| 33 | | SMN2.7A(-246-227) | GCU CAU GCC UAC AAU CCC AC | 20 | 59 |
| 34 | | SMN2.7A(-240-221) | GCA GUG GCU CAU GCC UAC AA | 20 | 78 |
| 35 | | SMN2.7A(-228-209) | UAA GGU UUU CUU GCA GUG GC | 20 | 67 |
| 36 | | SMN2.7A(-210-191) | CAA UUA UUA GGC UGC AGU UA | 20 | 61 |
| 37 | | SMN2.7A(-196-177) | UAU CCC AAA GAA AAC AAU UA | 20 | 62 |
| 38 | | SMN2.7A(-176-157) | UUU UAA UGU ACU UUA AAA GU | 20 | 66 |
| 39 | | SMN2.7A(-170-151) | AUA GUC UUU UAA UGU ACU UU | 20 | 53 |
| 40 | | SMN2.7A(-150-131) | UAU GAU CAG AAA UUA AGU UG | 20 | 73 |
| 41 | | SMN2.7A(-138-119) | UAU UCA ACA AAA UAU GAU CA | 20 | 61 |
| | Intron 7 | | | | |
| 42 | | SMN2.7D(-130-154) | UUU UGG CAU CAA AAU UCU UUA AUA U | 25 | 72 |
| 43 | | SMN2.7D(-135-159) | AAU AGU UUU GGC AUC AAA AUU CUU U | 25 | 83 |
| 44 | | SMN2.7D(-135-162) | UCU AAU AGU UUU GGC AUC AAA AUU CUU U | 28 | 84 |
| 45 | | SMN2.7D(-140-169) | CCU UUU AUC UAA UAG UUU UGG CAU CAA AAU | 30 | SO |
| 46 | | SMN2.7D(-145-174) | AUU AAC CUU UUA UCU AAU AGU UUU GGC AUC | 30 | 69 |
| 47 | | SMN2.7D(-150-174) | AUU AAC CUU UUA UCU AAU AGU UUU G | 25 | 54 |
| 48 | | SMN2.7D(-155-174) | AUU AAC CUU UUA UCU AAU AG | 20 | 69 |
| 49 | | SMN2.7D(-155-177) | UAG AUU AAC CUU UUA UCU AAU AG | 23 | 70 |
| 50 | | SMN2.7D(-155-180) | AUG UAG AUU AAC CUU UUA UCU AAU AG | 26 | 79 |
| 51 | | SMN2.7D(-175494) | GAA UUC UAG UAG GGA UGU AG | 20 | 64 |
| 52 | | SMN2.7D(-249-268) | AAA AUG GCA UCA UAU CCU AA | 20 | 76 |
| 53 | | SMN2.7D(-249-273) | GAU AUA AAA UGG CAU CAU AUC CUA A | 25 | 85 |
| 54 | | SMN2.7D(-252-271) | UAU AAA AUG GCA UCA UAU CC | 20 | 89 |
| 55 | | SMN2.7D(-254-273) | GAU AUA AAA UGG CAU CAU AU | 20 | 82 |

TABLE 6
SEQ ID listing of antisense oligomers inducing SMN2 Exon 7 inclusion into transcript

| SEQ ID | | | | Length |
|---|---|---|---|---|
| NO: | | Co-ordinates | Sequence | (bases) |
| | Intron 7 | | | |
| 56 | | SMN2.7D(-107-121)-(169-182) | GGA UGU AGA UUA ACU UAC AUU AAC CUU UC | 29 |
| 57 | | SMN2.7D(-137-159)-150A > U | AAU AGU UUU GGC UUC AAA AUU CU | 23 |
| 58 | | SMN2.7D(-137-159)-146U > A/-155A > U | AAU AGU UUA GGC AUC AAU AUU CU | 23 |
| 59 | | SMN2.7D(-137-159)-158C > U | AAU AGU UUU GGC AUC AAA AUU UU | 23 |
| 60 | | SMN2.7D(-137-159)-150A > U/-158C > U | AAU AGU UUU GGC UUC AAA AUU UU | 23 |

TABLE 7
SEQ ID listing of new antisense oligomers to be tested for inducing SMN2
Exon 7 inclusion into transcript

| SEQ ID | | | | Length |
|---|---|---|---|---|
| NO: | Region | Co-ordinates | Sequence | (bases) |
| | Intron 6 | | | |
| 61 | | SMN2.7A(-304-265) | UGG AGC UUG ACA CCA CCC UG | 20 |
| 62 | | SMN2.7A(-294-275) | ACU UGA GAC CUG GAG CUU GA | 20 |
| 63 | | SMN2.7A(-284-265) | UAG GGG GAU CAC UUG AGA CC | 20 |
| 64 | | SMN2.7A(-274-255) | GAG GCG GAG GUA GGG GGA UC | 20 |

For any of the sequences of Tables 1 to 7, each of the uracil bases (U) may be thymine bases (T).

### 2: Induction of SMN+Int 7 Transcripts

### Targeting Exon 8 to Retain Exon 7 and Intron 7

The inventors have shown herein that AOs directed to SMN2 exon 8 result in the majority of transcripts retaining exon 7 and intron 7, thereby providing functional SMN2-derived transcripts. Type I SMN patient fibroblasts transfected with morpholino oligomers (AO SEQ ID NOs: 14-17) promoting the inclusion of exon/intron 7 exhibited increased SMN expression. Retention of intron 7 in the mature mRNA may compromise the stability of the SMN transcript by either altering polyadenylation, or introduction of destabilizing elements that may regulate translation.

### 3: Alternative Oligomer Design to Enhance SMN Exon 7 Inclusion

### Retention of Exon 7

Table 6 provides a number of modified AO sequences. AO SEQ ID NO: 56 describes a “stapling” AO, which binds at two areas either side of a silencer region. AO SEQ ID NOs: 57-60 describe mismatched AO sequences containing one or more mismatched bases to the RNA sequence (data not shown). Both of these strategies are predicted to alter RNA secondary structure and improve inclusion.

### REFERENCES

1. Prior T W, et al. Homozygous SMN1 deletions in unaffected family members and modification of the phenotype by SMN2. Am J Med Genet A. 2004 Oct. 15; 130A(3):307-10.
2. Liu Q, Dreyfuss G. A novel nuclear structure containing the survival of motor neurons protein. EMBO J. 1996 Jul. 15; 15(14):3555-65.
3. Meister G, et al. A multiprotein complex mediates the ATP-dependent assembly of spliceosomal U snRNPs. Nat Cell Biol. 2001 November; 3(11):945-9.
4. Pellizzoni L, et al. Essential role for the SMN complex in the specificity of snRNP assembly. Science. 2002 Nov. 29; 298(5599):1775-9.
5. Gubitz A K, et al. The SMN complex. Exp Cell Res. 2004 May 15; 296(1):51-6.
6. Hua Y, Zhou J. Survival motor neuron protein facilitates assembly of stress granules. FEBS Lett. 2004 Aug. 13; 572(1-3):69-74.
7. Monani U R, et al. A single nucleotide difference that alters splicing patterns distinguishes the SMA gene SMN1 from the copy gene SMN2. Hum Mol Genet. 1999 July; 8(7):1177-83.
8. Hua Y, et al. Enhancement of SMN2 exon 7 inclusion by antisense oligonucleotides targeting the exon. PLoS Biol. 2007 April; 5(4):e73.
9. Singh RN. Evolving concepts on human SMN pre-mRNA splicing. RNA Biol. 2007 January-March; 4(1):7-10.
10. Hua Y, et al. Antisense masking of an hnRNP A1/A2 intronic splicing silencer corrects SMN2 splicing in transgenic mice. Am J Hum Genet. 2008 April; 82(4):834-48.
11. Lim S R, Hertel K J. Modulation of survival motor neuron pre-mRNA splicing by inhibition of alternative 3 splice site pairing. J Biol Chem. 2001 Nov. 30; 276(48):45476-83.
12. Swoboda K J, et al. Natural history of denervation in SMA: relation to age, SMN2 copy number, and function. Ann Neurol. 2005 May; 57(5):704-12.
13. Monani U R, et al. Animal models of spinal muscular atrophy. Hum Mol Genet. 2000 October; 9(16):2451-7.
14. Burghes A H. When is a deletion not a deletion? When it is converted. Am J Hum Genet. 1997 July; 61(1):9-15.
15. Schrank B, et al. Inactivation of the survival motor neuron gene, a candidate gene for human spinal muscular atrophy, leads to massive cell death in early mouse embryos. Proc Natl Acad Sci USA. 1997 Sep. 2; 94(18):9920-5.
16. Andreassi C, et al. Aclarubicin treatment restores SMN levels to cells derived from type 1 spinal muscular atrophy patients. Hum Mol Genet. 2001 Nov. 15; 10(24):2841-9.
17. Chang J G, et al. Treatment of spinal muscular atrophy by sodium butyrate. Proc Natl Acad Sci USA. 2001 Aug. 14; 98(17):9808-13.
18. Brichta L, et al. Valproic acid increases the SMN2 protein level: a well-known drug as a potential therapy for spinal muscular atrophy. Hum Mol Genet. 2003 Oct. 1; 12(19):2481-9.
19. Sumner C J, et al. Valproic acid increases SMN levels in spinal muscular atrophy patient cells. Ann Neurol. 2003 November; 54(5):647-54.
20. Kernochan L E, et al. The role of histone acetylation in SMN gene expression. Hum Mol Genet. 2005 May 1; 14(9):1171-82.
21. Singh J, et al. DcpS as a therapeutic target for spinal muscular atrophy. ACS Chem Biol. 2008 Nov. 21; 3(11):711-22.
22. Lorson C L, Androphy E J. An exonic enhancer is required for inclusion of an essential exon in the SMA-determining gene SMN. Hum Mol Genet. 2000 Jan. 22; 9(2):259-65.
23. Cartegni L, Krainer A R. Correction of disease-associated exon skipping by synthetic exon-specific activators. Nat Struct Biol. 2003 February; 10(2):120-5.
24. Skordis L A, et al. Bifunctional antisense oligonucleotides provide a trans-acting splicing enhancer that stimulates SMN2 gene expression in patient fibroblasts. Proc Natl Acad Sci USA. 2003 Apr. 1; 100(7):4114-9.
25. Madocsai C, et al. Correction of SMN2 Pre-mRNA splicing by antisense U7 small nuclear RNAs. Mol Ther. 2005 December; 12(6):1013-22.
26. Lorson C L, et al. A single nucleotide in the SMN gene regulates splicing and is responsible for spinal muscular atrophy. Proc Natl Acad Sci USA. 1999 May 25; 96(11):6307-11.
27. Errington S J, et al. Target selection for antisense oligonucleotide induced exon skipping in the dystrophin gene. J Gene Med. 2003 June; 5(6):518-27.
28. Singh N K, et al. Splicing of a critical exon of human Survival Motor Neuron is regulated by a unique silencer element located in the last intron. Mol Cell Biol. 2006 February; 26(4):1333-46.
29, Gebski B L, et al. Morpholino antisense oligonucleotide induced dystrophin exon 23 skipping in mdx mouse muscle, Hum Mol Genet. 2003 Aug. 1; 12(15):1801-11.
30. Baughan T, et al. Stimulating full-length SMN2 expression by delivering bifunctional RNAs via a viral vector. Mol Ther. 2006 July; 14(1):54-62.
31. Coady T H, et al. Development of a single vector system that enhances trans-splicing of SMN2 transcripts. PLoS ONE. 2008; 3(10):e3468.
32. Dickson A. et al. A Negatively-Acting Bifunctional RNA Increases Survival Motor Neuron in vitro and in vivo. Hum Gene Ther. 2008 Aug. 25.
33. Jearawiriyapaisarn N, et al. Sustained Dystrophin Expression Induced by Peptide-conjugated Morpholino Oligomers in the Muscles of mdx Mice. Mol Ther. 2008 Jun. 10.
34. Baughan T D, et al. Delivery of bifunctional RNAs that target an intronic repressor and increase SMN levels in an animal model of spinal muscular atrophy. Hum Mol Genet. 2009 Feb. 19.
35. Bentley D. The mRNA assembly line: transcription and processing machines in the same factory. Curr Opin Cell Biol. 2002 June; 14(3):336-42.
36. Bentley D L. Rules of engagement: co-transcriptional recruitment of pre-mRNA processing factors. Curr Opin Cell Biol. 2005 June; 17(3):251-6.
37. Monani U R, et al. The human centromeric survival motor neuron gene (SMN2) rescues embryonic lethality in SMN(−/−) mice and results in a mouse with spinal muscular atrophy. Hum Mol Genet. 2000 Feb. 12; 9(3):333-9.
38. Mann C J, et al. Improved antisense oligonucleotide induced exon skipping in the mdx mouse model of muscular dystrophy. J Gene Med. 2002 November-December; 4(6):644-54.
39. Coovert D D, et al. The survival motor neuron protein in spinal muscular atrophy. Hum Mol Genet. 1997 August; 6(8):1205-14.
40. Young P J, et al. The relationship between SMN, the spinal muscular atrophy protein, and nuclear coiled bodies in differentiated tissues and cultured cells. Exp Cell Res. 2000 May 1; 256(2):365-74.
41. Cooper S T, et al. Single section Western blot: improving the molecular diagnosis of the muscular dystrophies. Neurology. 2003 Jul. 8; 61(1):93-7.
42. Yin H, et al. Effective exon skipping and restoration of dystrophin expression by peptide nucleic acid antisense oligonucleotides in mdx mice. Mol Ther. 2008 January; 16(1):38-45.
43. Monani U R, et al. A transgene carrying an A2G missense mutation in the SMN gene modulates phenotypic severity in mice with severe (type I) spinal muscular atrophy. J Cell Biol. 2003 Jan. 6; 160(1):41-52.
44. Avila A M, et al. Trichostatin A increases SMN expression and survival in a mouse model of spinal muscular atrophy. J Clin Invest. 2007 March; 117(3):659-71.
45. Narver H L, et al. Sustained improvement of spinal muscular atrophy mice treated with trichostatin A plus nutrition. Ann Neurol. 2008 October; 64(4):465-70.
46. Pennisi E. Genetics. Hopping to a better protein. Science. 2008 Dec. 5; 322(5907):1454-5.

1. An antisense oligonucleotide of 10 to 50 nucleotides comprising a targeting sequence complementary to a region near or within intron 6, intron 7, or exon 8 of the Survival Motor Neuron 2 (SMN2) gene pre-mRNA.

2. The antisense oligonucleotide of claim 1 wherein the antisense oligonucleotide is a phosphorodiamidate morpholino oligomer.

3. The antisense oligonucleotide of claim 1, wherein the targeting sequence is selected from any of Tables 1, 2 and 5 to 7.

4. The antisense oligonucleotide of claim 1, wherein the targeting sequence is selected from SEQ ID NOS: 7 to 17 and 29 to 64.

5. The antisense oligonucleotide of claim 4, wherein the targeting sequence is selected from SEQ ID NOS: 7 to 13, 29, 44, 53 and 54.

6. The antisense oligonucleotide of claim 4, wherein the targeting sequence is selected from SEQ ID NOS: 7 to 13.

7. The antisense oligonucleotide of claim 4, wherein the targeting sequence is selected from SEQ ID NO: 10.

8. The antisense oligonucleotide of claim 4, wherein the oligonucleotide comprises 15 to 30 nucleotides.

9. The antisense oligonucleotide of claim 3, wherein the oligonucleotide comprises 25 to 30 nucleotides.

10. A pharmaceutical composition, comprising:
an antisense oligonucleotide of 10 to 50 nucleotides comprising a targeting sequence complementary to a region near or within intron 6, intron 7, or exon 8 of the Survival Motor Neuron 2 (SMN2) gene pre-mRNA; and
a pharmaceutically acceptable carrier.

11. The pharmaceutical composition of claim 10 wherein the antisense oligonucleotide is a phosphorodiamidate morpholino oligomer.

12. The pharmaceutical composition of claim 10, wherein the targeting sequence is selected from any of Tables 1, 2 and 5 to 7.

13. The pharmaceutical composition of claim 10, wherein the targeting sequence is selected from SEQ ID NOS: 7 to 17 and 29 to 64.

14-16. (canceled)

17. The pharmaceutical composition of claim 13, wherein the oligonucleotide comprises 15 to 30 nucleotides.

18. (canceled)

19. A method of treating Spinal muscular atrophy (SMA) or a condition associated with SMA in a subject in need thereof, comprising administering to the subject an effective amount of an antisense oligonucleotide of 10 to 50 nucleotides comprising a targeting sequence complementary to a region near or within intron 6, intron 7, or exon 8 of the Survival Motor Neuron 2 (SMN2) gene pre-mRNA.

20. The method of claim 19 wherein the antisense oligonucleotide is a phosphorodiamidate morpholino oligomer.

21. The method of claim 19, wherein the targeting sequence is selected from any of Tables 1, 2 and 5 to 7.

22. The method of claim 19, wherein the targeting sequence is selected from SEQ ID NOS: 7 to 17 and 29 to 64.

23-25. (canceled)

26. The method of claim 22, wherein the oligonucleotide comprises 15 to 30 nucleotides.

27-36. (canceled)
